(19) United States
(12) Patent Application Publication
(10) Pub. No.: US 2011/0137075 A1
(43) Pub. Date: Jun. 9, 2011

(54) Vinyl acetate production process

(76) Inventors: Wayne J. Brtko, Glen Mills, PA (US); Brian A. Salisbury, Oxford, PA (US); Noel Hallinan, Loveland, OH (US)

(21) Appl. No.: 12/653,144

(22) Filed: Dec. 9, 2009

(51) Int. Cl. C07C 67/00

(52) U.S. Cl. 560/261

(57) ABSTRACT

A process for the production of vinyl acetate is disclosed. The process comprises reacting ethylene, acetic acid, and oxygen in the presence of a catalyst to produce a reaction mixture comprising vinyl acetate, ethylene, carbon dioxide, acetic acid, water and ethylene glycol diacetate. The reaction mixture is separated to a gas stream comprising ethylene, and carbon dioxide and a crude vinyl acetate stream comprising vinyl acetate, acetic acid, water and ethylene glycol diacetate. An ethylene glycol diacetate stream is isolated from the crude vinyl acetate stream and subjected to a reactive distillation to recover acetic acid.

### FIELD OF THE INVENTION

The invention relates to the preparation of vinyl acetate. More particularly, the invention relates to recovering acetic acid from ethylene glycol diacetate.

### BACKGROUND OF THE INVENTION

Vinyl acetate is commonly produced by the reaction of ethylene, oxygen and acetic acid in the presence of a palladium-gold catalyst. See, for example, U.S. Pat. No. 3,743,607. Palladium and gold are expensive precious metals. Therefore, many efforts have been made to increase the catalytic activity and reduce the amount of catalyst needed. For example, U.S. Pat. No. 6,022,823 teaches calcining the support impregnated with palladium and gold salts prior to reducing the metals. The catalyst shows improved activity.

The acetoxylation of ethylene to vinyl acetate is commonly performed in a gas phase, fixed bed tubular reactor. Vinyl acetate is recovered by condensation and scrubbing, and purified by distillation. Unreacted ethylene, oxygen and acetic acid are recovered by distillation and recycled to the acetoxylation.

In addition to vinyl acetate, the acetoxylation produces a number of byproducts, including carbon dioxide, water, and ethylene glycol diacetate. Carbon dioxide is primarily produced by the combustion of ethylene and vinyl acetate. Carbon dioxide is removed from the reaction product mixture by distillation and absorption with a potassium carbonate solution. The acetoxylation produces from 1 to 2 pounds of ethylene glycol diacetate.

Copending U.S. Pat. Appl. Ser. No. 12/587,580, filed on Sep. 9, 2009, discloses a process for the production of vinyl acetate. The process comprises reacting ethylene, acetic acid, and oxygen in the presence of a catalyst to produce a reaction mixture comprising vinyl acetate, ethylene, carbon dioxide, acetic acid and ethylene glycol diacetate. The reaction mixture is separated to a gas stream comprising ethylene, oxygen, and carbon dioxide and a crude vinyl acetate stream comprising vinyl acetate, acetic acid and ethylene glycol diacetate. An ethylene glycol diacetate stream is isolated from the crude vinyl acetate stream and hydrolyzed to recover acetic acid.

New method for recovering acetic acid from the vinyl acetate production is needed.

### SUMMARY OF THE INVENTION

The invention is a process for the production of vinyl acetate. The process comprises reacting ethylene, acetic acid and oxygen in the presence of a catalyst to produce a reaction mixture comprising vinyl acetate, ethylene, carbon dioxide, acetic acid, water and ethylene glycol diacetate. The reaction mixture is separated to a gas stream comprising ethylene and carbon dioxide and a crude vinyl acetate stream comprising vinyl acetate, acetic acid, water and ethylene glycol diacetate. An ethylene glycol diacetate stream is isolated from the crude vinyl acetate stream and subjected to a reactive distillation to recover acetic acid which is preferably is recycled to an acetic acid or vinyl acetate process.

### DETAILED DESCRIPTION OF THE INVENTION

The process of the invention comprises reacting ethylene, acetic acid, and oxygen in the presence of a catalyst. The acetoxylation is preferably performed in a gas phase, fixed bed tubular reactor using a supported catalyst. Preferably, the acetoxylation is performed at a temperature within the range of 150° C. to 250° C., more preferably 175° C. to 200° C. Preferably, the acetoxylation is performed under a pressure within the range of 50 psia to 150 psia, and more preferably within the range of 70 psia to 140 psia.

Preferably, the amount of oxygen in the combined feed is within the range of 5 mol % to 15 mol %, more preferably within the range of 5 mol % to 12 mol %. Acetic acid may be introduced into the reactor in liquid form or in vapor form. Preferably, the amount of acetic acid in the combined feed is within the range of 10 mol % to 25 mol. Preferably, the amount of ethylene in the combined feed is within the range of 65 mol % to 80 mol %. Preferably, ethylene, oxygen and acetic acid are mixed and the mixture is then fed into the reactor as a gas.

Suitable catalysts include those known to the vinyl acetate industry. Preferably, the catalyst is a palladium-gold catalyst. Methods for preparing palladium-gold catalysts are known. For instance, U.S. Pat. No. 6,022,823, the teachings of which are incorporated herein by reference, teaches how to prepare a palladium-gold catalyst which has high activity and selectivity. Preferably, the palladium-gold catalyst is supported on an inorganic oxide. Preferably, the inorganic oxide is selected from the group consisting of alumina, silica, titania, the like, and mixtures thereof.

Preferably, the supported catalysts have palladium contents from 0.1 wt % to 3 wt % and gold contents from 0.1 wt % to 3 wt %. More preferably, the catalysts contain from 0.5 wt % to 1.5 wt % of palladium and from 0.25 wt % to 0.75 wt % of gold. The weight ratio of palladium to gold is preferably within the range of 5:1 to 1:3 and more preferably within the range of 2.5:1 to 1:1.5.

The reaction mixture is withdrawn from the reactor and separated into a gas stream and a crude vinyl acetate stream. The gas stream comprises ethylene, and carbon dioxide. The crude vinyl acetate stream comprises vinyl acetate, acetic acid, water and ethylene glycol diacetate. Carbon dioxide is separated by distillation or absorption from ethylene which is then preferably recycled to the acetoxylation reactor. The crude vinyl acetate stream is separated by distillation into an ethylene glycol diacetate stream which comprises ethylene glycol diacetate, acetic acid and water and a vinyl acetate product stream which comprises vinyl acetate. The vinyl acetate product stream may be subjected to further purification to produce vinyl acetate with a desired purity.

The ethylene glycol diacetate stream may also comprise other components such as ethylene glycol and polyvinyl acetate. The ethylene glycol diacetate stream preferably comprises at least 1 wt % of ethylene glycol diacetate. More preferably, the ethylene glycol diacetate stream comprises from 1 wt % to 50 wt % of ethylene glycol diacetate. Most preferably, the ethylene glycol diacetate stream comprises from 2 wt % to 20 wt % of ethylene glycol diacetate.

The ethylene glycol diacetate stream undergoes a reactive distillation. The reactive distillation is performed in the presence of a hydrolysis catalyst. Suitable hydrolysis catalysts include base and acid catalysts. Examples of base catalysts include ammonia, organic amines, metal hydroxides, basic ion-exchange resins, the like, and mixtures thereof. Examples of acid catalysts include sulfuric acid, sulfonic acids, acidic ion-exchange resins, the like, and mixtures thereof. Basic or acidic ion-exchange resins are preferred.

The reactive distillation column comprises a reaction section wherein the ethylene glycol diacetate is hydrolyzed. Preferably, the polyvinyl acetate is also hydrolyzed. The hydrolysis catalyst is placed in the reaction section. The reactive distillation column preferably has 8 to 25 theoretical stages. Preferably, the reaction section comprises 3 theoretical stages in the middle of the column. The ethylene glycol diacetate stream is preferably fed to the middle of the reaction section. Optionally, additional amount of water is fed together with the ethylene glycol diacetate stream. Alternatively, additional water can be fed to a stage above the reaction section. The reactive distillation column overhead stream is preferably taken at a pressure within the range of 20 psia to 30 psia and a temperature within the range of 38° C. to 52° C. The overhead stream comprises water and acetic acid and it is preferably recycled to an acetic acid or vinyl acetate process for recovery or reuse of acetic acid. The reactive distillation column bottoms stream is preferably taken at a pressure within the range of 20 psia to 30 psia and a temperature within the range of 205° C. to 218° C. This bottoms stream comprises acetic acid, ethylene glycol and polyvinyl acetate or polyvinyl alcohol and can be subjected to further separation or disposed of as waste.

The following example is merely illustrative. Those skilled in the art will recognize many variations that are within the spirit of the invention and scope of the claims.

### EXAMPLE

This example is modeled by a computer program. An ethylene glycol diacetate stream (100 parts by weight) from a vinyl acetate process is fed to a reactive distillation column containing a base catalyst. The stream comprises water 1%, acetic acid 94%, ethylene glycol diacetate 4.5%, and polyvinyl acetate 0.5%. The water feed tray is directly above the section of the column containing the base catalyst. The distillation column contains 8 theoretical trays. The ethylene glycol diacetate stream is fed at theoretical tray 6 from the top. Water (3.5 parts by weight) is fed at theoretical tray 4 from the top.

The column overhead stream (101 parts by weight), which comprises 3.5% water and 96.5% acetic acid, is taken at 21 psia and 38° C. Acetic acid recovery based on the amount in the feed is 104% because of the acetic acid produced in the hydrolysis reaction. This overhead stream is recycled to an acetic acid or dried and then recycled to the vinyl acetate process. The column bottoms stream (2.5 parts by weight) comprises less than 1 ppm of water, 1.9% acetic acid, 77.8% ethylene glycol, and 20.3% polyvinyl acetate. This stream is taken at 20.7 psia and 206° C.

We claim:

1. A process for the production of vinyl acetate comprising: (a) reacting ethylene, acetic acid, and oxygen in the presence of a catalyst to produce a reaction mixture comprising vinyl acetate, ethylene, carbon dioxide, acetic acid, water and ethylene glycol diacetate; (b) separating the reaction mixture to form a gas stream comprising ethylene and carbon dioxide and a crude vinyl acetate stream comprising vinyl acetate, acetic acid, water and ethylene glycol diacetate; (c) isolating an ethylene glycol diacetate stream which comprises ethylene glycol diacetate, acetic acid and water from the crude vinyl acetate stream; and (d) subjecting the ethylene glycol diacetate stream to a reactive distillation in the presence of a hydrolysis catalyst to hydrolyze the ethylene glycol diacetate and to form an overhead stream comprising acetic acid and water and a bottoms stream comprising ethylene glycol.

2. The process of claim 1, wherein the hydrolysis catalyst is selected from the group consisting of ammonia, organic amines, metal hydroxides, basic ion-exchange resins, sulfuric acid, sulfonic acids, and acidic ion-exchange resins.

3. The process of claim 1, wherein the overhead stream from step (d) is subjected to distillation to remove water from acetic acid.

4. The process of claim 3, wherein the resultant acetic acid is recycled to step (a).

5. The process of claim 1, wherein step (d) is performed in a reactive distillation column having 8 to 25 theoretical stages.

6. The process of claim 5, wherein the hydrolysis catalyst is placed in the middle of the reactive distillation column.

7. The process of claim 6, wherein additional water is added in step (d).

8. The process of claim 7, wherein the additional water is added in the stage where the hydrolysis catalyst is located or a stage above where the hydrolysis catalyst is located.

9. The process of claim 1, wherein the overhead stream is recycled to an acetic acid production process.
